(19) United States
(12) Patent Application Publication
(10) Pub. No.: US 2008/0064024 A1
(43) Pub. Date: Mar. 13, 2008

(54) Methods and apparatus for enzyme inhibitors analysis

(75) Inventors: Yueh-Hui Lin, Hsinchu (TW); Thomas Y.S. Shen, Hsinchu (TW)

Correspondence Address: REED SMITH LLP, 3110 FAIRVIEW PARK DRIVE, SUITE 1400, FALLS CHURCH, VA, 22042, US

(73) Assignee: Apex Biotechnology Corp.

(21) Appl. No.: 11/802,381

(22) Filed: May 22, 2007

(30) Foreign Application Priority Data
May 26, 2006 (TW) 95118875

(51) Int. Cl. C12Q 1/00; C12M 1/00

(52) U.S. Cl. 435/4; 435/287.1

(57) ABSTRACT

Methods and Apparatus for Enzyme Inhibitors Analysis The present invention provides apparatus and methods for determining an amount of an enzyme inhibitor in an aqueous solution, wherein an electrode strip and an electroanalysis meter are utilized, and the electrode strip can only be used one time. The present invention can also be applied to determine pesticides remained within the vegetables and fruits.

### CROSS REFERENCE TO RELATED APPLICATIONS

This application claims the right of priority based on Taiwan Patent Application No. 095118875 entitled “METHODS AND APPARATUS FOR ENZYME INHIBITORS ANALYSIS,” filed on May 26, 2006, which is incorporated herein by reference and assigned to the assignee herein.

1. Field of the Invention

The present invention relates to an apparatus and a method for determining an amount of an enzyme inhibitor, and more particularly, relates to an electrochemical analysis method for determining the type of an enzyme inhibitor within a pesticide.

2. Background of the Invention

There are many methods for determining the amount of pesticides remained within the vegetables and fruits in the prior art, such as spectrophotometry, atomic absorption spectrometry, thin-layer chromatography, gas chromatography, liquid chromatography, NMR spectroscope, fluorescence, etc. Among these methods, the gas chromatography and liquid chromatography are most common because they are good in reproducibility and sensitivity, and moreover, in determining the type of pesticides. However, these methods need a lot of samples prepared by professional technicians in laboratories, and the untrained users can not easily and quickly determine the amount or the type of residual pesticides in a shopping place, such as a crop filed, a farmers' market or a supermarket.

In recent years, biochemical reaction and electrochemical techniques have been used in determining what type of enzyme inhibitors within pesticides, such as U.S. Pat. No. 6,406,876 by Gardon et al. and the thesis by Marazuela et al. (M. D. Marazuela, M. C. Moreno-Bondi, Anal. Chim. Acta, 374 (1998) 19) disclosed methods of immobilized enzyme technology without sophisticated sample preparation. However, the immobilized enzyme methods are cumbersome processes with high cost, and the maintenance of electrodes requires strict care. Accordingly, these methods have to be performed by professional technicians.

Therefore, there is a need to provide a novel apparatus and a method to overcome the limitations, and enable unprofessional users to conveniently use it everywhere.

### SUMMARY OF THE INVENTION

According to characteristics of inhibiting an enzyme activity of pesticides, the present invention provides an apparatus and a method for determining an amount of an enzyme inhibitor conveniently so as to determine an amount of pesticides within a sample.

The present invention provides a first enzyme for catalyzing a biochemical reaction of a first compound. An enzyme inhibitor is capable of inhibiting the catalytic reaction of the first enzyme, so by observing a variation of an inhibited degree of the first enzyme (a degree for slowing the biochemical reaction), an amount of the enzyme inhibitor can be determined. The step of determining the inhibited degree of the first enzyme inhibitor includes providing a third compound to form a fourth compound by reacting the third compound with the product (i.e. a second compound) generated from the biochemical reaction. The fourth compound is available for an electrochemical reaction. Then, the amount of the enzyme inhibitor is determined according to the degree of slowing the biochemical reaction represented in the variation of an electric current generated by the fourth compound.

One aspect of the present invention is to provide a method for determining an amount of an enzyme inhibitor within a sample. The method includes: (a) providing a first enzyme having a dosage for one time use; (b) providing a reference sample including the enzyme inhibitor of a given amount, the enzyme inhibitor capable of inhibiting a catalytic reaction of the first enzyme; (c) providing an electrode strip coated with reactants having a dosage for one time use, the reactants including a first compound; (d) mixing the first enzyme and the reference sample to form a solution, and then applying the solution to the electrode strip to contact the first enzyme with the first compound; (e) applying a voltage to the electrode strip, and measuring a reference electric current; (f) providing the sample, and using the sample instead of the reference sample to repeat steps (a) to (e) and obtain an electric current; and (g) determining the amount of the enzyme inhibitor within the sample according to the reference electric current and the electric current.

Another aspect of the present invention is to provide an apparatus for determining an amount of an enzyme inhibitor within a sample. The apparatus includes an electrode strip and a microprocessor. The electrode strip includes an insulation substrate and reactants having a dosage for one time use on the insulation substrate. The microprocessor couples with the electrode strip. When the sample and a first enzyme contact with the first compound, an electric signal is generated, and the microprocessor processes the electric signal to determine the amount of the enzyme inhibitor inhibiting the first enzyme.

The present invention includes using dry strips having a dosage for one time use such that the preparation of many kinds of sample solutions can be omitted. Moreover, the method and the apparatus for determining an amount of an enzyme inhibitor of the present invention further analyze whether the amount of pesticides remained within a sample exceeds the criterion.

### BRIEF DESCRIPTION OF THE DRAWINGS

FIG. 1 illustrates an embodiment of the present invention.

FIG. 2 illustrates an embodiment including a temperature control component of the present invention.

FIG. 3 illustrates an embodiment including a temperature compensation module of the present invention.

FIG. 4 shows a relationship between the concentration of an enzyme inhibitor and the activity determined in accordance with the method of the present invention.

### DETAILED DESCRIPTION OF THE INVENTION

As described above, the present invention provides an apparatus and a method for determining an amount of an enzyme inhibitor in an aqueous solution. In general, pesticides include enzyme inhibitors, and accordingly the present invention may be applied to determine an amount of pesticides within a sample. In other words, the present invention may apply to analyze pesticides including enzyme inhibitors, wherein the enzyme inhibitors may include but not limited to organophosphorus, thio-organophosphorus treated by a bromide water or carbamate.

The following examples are intended to illustrate the chemical reagents and the reaction principles of the present invention, and the examples may be understood to illustrate but not to limit the scope of the invention.

The chemical reagents are examples employed in the present invention including:

First compound (I): a reactant in a hydrolysis reaction, such as acetylcholine (ACh) or acetylthiocholine (ATC).

Second compound (II): a hydrolyte of the first compound (I), wherein if the first compound I is ACh, the second compound (II) may be choline as the description by D. M. Ivnitskii and J. Rishpon in Biosensor & Bioelectronics 8 (1993) 265-271, which is incorporated herein by reference. If the first compound (I) is acetylthiocholine (ATC), the second compound (II) may be thiocholine, as the description by D. M. Ivnitskii and J. Rishpon in Biosensor & Bioelectronics 9 (1994) 569-576, which is also incorporated herein by reference.

First enzyme (E₁): capable of catalyzing a hydrolysis reaction of the first compound (I), wherein an example of the first enzyme (E₁) is acetylcholine esterase (AChE).

Third compound (III): a reactant, an oxidant generally, capable of oxidizing the second compound (II) (i.e. a product of a hydrolysis reaction) to produce a fourth compound (IV) and betaine, wherein an example of the third compound (III) is oxygen (O₂) or ferric cyanide (Fe(CN)₆³⁻).

Fourth compound (IV): capable of generating electrons, namely applying a suitable voltage to the fourth compound (IV) may generate an electric current. For example, if the third compound (III) is oxygen (O₂), the fourth compound (IV) may be hydrogen peroxide (H₂O₂); or if the third compound (III) is ferric cyanide (Fe(CN)₆³⁻), the fourth compound (IV) may be ferrocyanide (Fe(CN)₆⁴⁻).

Second enzyme (E₂): capable of catalyzing an oxidation reaction of the second compound (II), wherein an example of the second enzyme (E₂) is choline oxidase (ChOx). The second enzyme (E₂) is optional. In other words, the second enzyme (E₂) is employed when an oxidation rate is too slow causing the concentration of the second compound (II) to significantly increase versus time.

The reactions involved in the embodiments of the present invention are shown below:

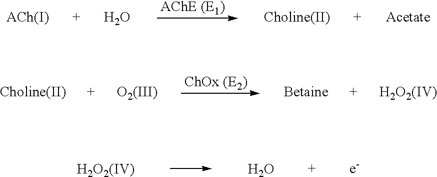

If the above-mentioned first compound (I) is acetylthiocholine (ATC), the third compound (III) may be ferric cyanide (Fe(CN)₆³⁻) instead. The chemical reactions are shown below:

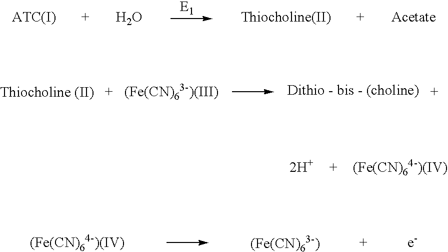

According to the foregoing reaction equations, a suitable recovery voltage may be applied to the fourth compound so as to generate an electric current. Subsequently, a formation rate of the fourth compound (IV) is determined according to a variation in the electric current. As above-mentioned, the formation rate of the fourth compound (IV) is substantially equal to the formation rate of the second compound (II) due to the oxidation-reduction reaction involving the second compound (II) oxidizing with the third compound (III) to produce the fourth compound (IV) is very fast. Thus, if a reference sample with the first compound (I) of a given concentration is provided, the reference formation rate of the second compound (II) can be obtained. Accordingly, when an unknown sample with a first enzyme inhibitor is tested, the formation rate of the second compound (II) obtained will be lower than the reference value because the first enzyme inhibitor is capable of inhibiting a catalytic reaction of the first enzyme (E₁). Therefore, the amount of the first inhibitor within the unknown sample is determined based on the variation in the formation rate of the second compound (II) (i.e. the variation in the formation rate of the fourth compound (IV)). It should be noted that the enzyme inhibitor in the present invention is direct to the first enzyme (E₁) not that the second enzyme (E₂).

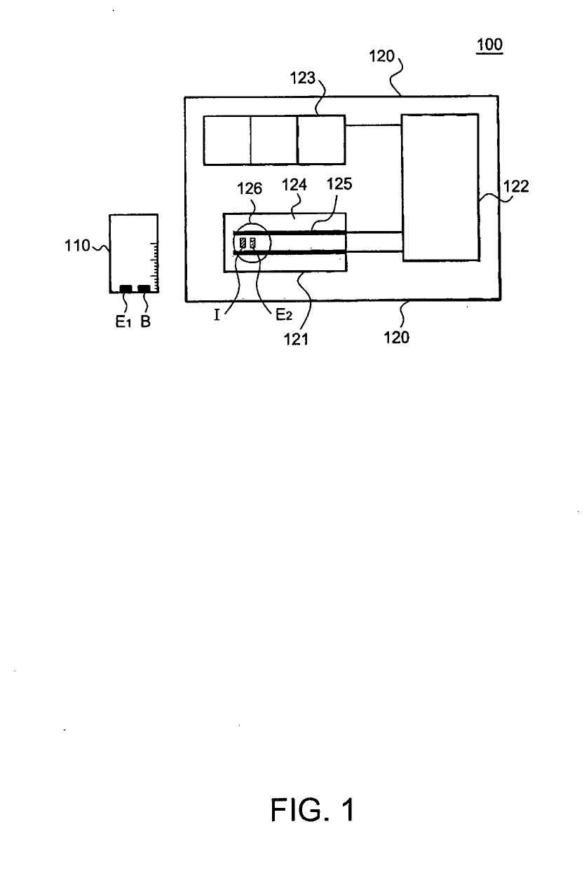

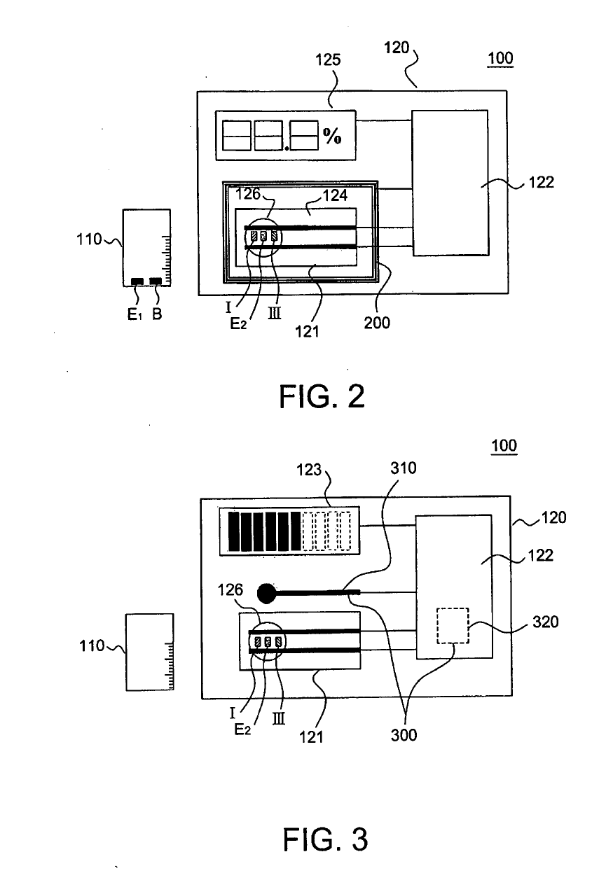

The principle of the present invention is illustrated above. Apparatus and methods of the present invention are illustrated in detail below. As shown in FIG. 1, an analysis apparatus 100 includes a container 110 and an electroanalysis meter 120. The container 110 is for containing a sample to be determined. An inner surface of the container 110 can be coated with a first enzyme (E₁) having a dosage for one time use optionally. The electroanalysis meter 120 includes an electrode strip 121, a microprocessor 122, and a monitor 123. The electrode strip 121 includes an insulation substrate 124, an electrode system 125 on the insulation substrate 124, and a reaction area 126 coated with the first compound (I) having a dosage for one time use and the second enzyme (E₂). The third compound (III) may be optionally coated on the reaction area 126 (as shown in FIG. 2 and FIG. 3). The reaction area 126 connects to the electrode system 125, the electrode system 125 connects to the microprocessor 122, and the microprocessor 122 connects to the monitor 123. Thus, when users apply the first enzyme (E₁) to the reaction area 126, the first compound (I) contacts with the first enzyme (E₁) to initiate the above-mentioned reactions, and an electric signal is then generated. The electrode strip 125 passes the electric signal to the microprocessor 122 for processing, and the result is then shown on the monitor 123. It should be noted that the foregoing chemical reagents may be coated on the inner surface of the container 110 or the surface of the insulation substrate 124, and moreover, the chemical reagents are dry and have a dosage for one time use, respectively, before reacting. The electrode strip 121 is for one time use and to be thrown away after reacting. The chemical reagents in the container 110 or on the electrode strip 121 are not limited to those mentioned above. Users may optionally add other additives, such as an additive, a buffer salt, a surfactant, and the like. The additives can be coated on the inner surface of the container 110, such as the buffer salt (B) shown in the FIG. 1.

Furthermore, the third compound (III) can be in solid phase, such as ferric cyanide, or in gas phase, such as oxygen. When the third compound (III) is oxygen, which may be provided from the atmosphere under the atmospheric environment balance. Alternatively, oxygen can be provided through a delivery pipe as appropriate.

FIG. 2 shows the analysis apparatus 100 of the present invention including a temperature control component 200 for controlling the temperature around the reaction area 126 so that the reaction can be performed at a constant temperature. FIG. 3 shows the analysis apparatus 100 of the present invention further including a temperature compensation module 300. The temperature module 300 includes a thermometer 310 and a temperature compensation processing firmware 320 integrated within the microprocessor 122. The temperature compensation module 300 may calculate the difference between the optimal reaction temperature and the practical temperature of the chemical reaction mentioned above, and subsequently, the deviation determined based on the difference is shown on the monitor 123.

The analysis method of the present invention includes the following steps.

(a) Providing a reference sample including the first enzyme inhibitor of a given amount, wherein the given amount includes zero.

(b) Mixing the reference sample with the first enzyme (E₁) to form an aqueous solution. For example, the reference sample may be placed in a container coated with the first enzyme (E₁) and mixed uniformly. Alternatively, the first enzyme (E₁) may be provided in another package, such as encapsulated in a capsule, and then mixed with the reference sample in the container. The first enzyme (E₁) has a dosage for one time use.

(c) Applying the aqueous solution to react with the electrode strip 121. As aforementioned, the electrode strip 121 may be coated with the first compound (I), and optionally with the second enzyme (E₂) or the third compound (III), which are provided in dry form. Namely, when the first compound (I) is ACh, the preferable second enzyme (E₂) and the third compound (III) is respectively ChOx and oxygen. In such a situation, the needed oxygen is preferably obtained from the air under the atmospheric environment balance or more preferably provided through a delivery pipe. Alternatively, when the first compound is ACT, the preferable third compound is Fe(CN)₆³⁻. In such a situation, the second enzyme (E₂) is not necessary. Similarly, the dosage of the first compound (I) or the third compound (III) (such as Fe(CN)₆³⁻) on the electrode strip 121 can are provided for one time use only.

(d) Applying a predetermined voltage to the electrode strip 121. For example, if the fourth compound (IV) is H₂O₂, the applied voltage may be 700 mV. If the fourth compound (IV) is Fe(CN)₆⁴⁻, the applied voltage may be 300 mV.

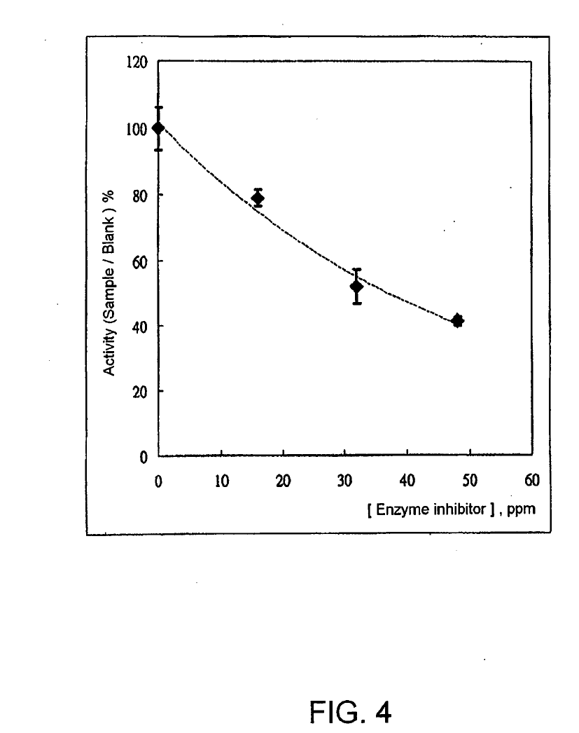

(e) Measuring a reference electric current generated by the fourth compound (IV). It should be noted that users may test multiple reference samples in different given amounts at certain temperatures so as to obtain the reference electric currents corresponding to the given amounts in various conditions for quantitative analysis. In practical, one may define an activity % as the electric current ratio of a reference sample to a blank test (i.e. the given amount is zero). FIG. 4 shows the relationship between the activities generated by the reference samples and the concentrations of the enzyme inhibitor at 25° C. The data of FIG. 4 can be stored in a memory device of the electroanalysis meter and retrieved by the microprocessor.

(f) Providing a sample including a first enzyme inhibitor of an unknown amount, wherein the sample may be from an aqueous solution that vegetable has been soaked in.

(g) Using the sample instead of the reference sample to repeat steps (a) to (d) so as to obtain an electric current generated by the fourth compound (IV).

(h) Determining the amount of the first enzyme within the sample according to the difference between the electric current and the reference electric current, wherein the electric current and the reference electric current may be stored in the microprocessor and processed to show the result on the monitor.

(i) Throwing away the electrode strip 121. It should be noted that the electrode strip 121 is for one time use and can not be reused. If the container 110 contains the one-time-use first enzyme (E₁), the container 110 should also be discarded and not to be reused.

The following examples are intended to illustrate a method for using the apparatus of the present invention by general users. In other words, the present invention can be used by a person without chemical analysis background.

### EXAMPLE 1

As shown in FIG. 1, an unknown sample is poured into the container 110 and shook adequately to form a well-mixed aqueous solution. Subsequently, the electroanalysis meter 120 is turned on, and the aqueous solution is provided on the reaction area 126 of the electrode strip 121. Then, the electroanalysis meter 120 applies a suitable voltage automatically and measures the electric current caused by the reaction. The microprocessor 122 determines the amount of the pesticide within the unknown sample and shows the quantitative result on the monitor 123. After the test is completed, the used electrode strip 121 is thrown away.

### EXAMPLE 2

As shown in FIG. 3, an unknown sample is poured into the container 110. The AChE and the buffer salt (B) packaged in a capsule with a dosage for one time use are poured into the container 110 too, and the container 110 is then shaken adequately to form a well-mixed aqueous solution. The electroanalysis meter 120 is turned on, and the aqueous solution is provided on the reaction area 126 of the electrode strip 121. The electroanalysis meter 120 will apply a suitable voltage automatically and measure the electric current caused by the reaction. Meanwhile, the microprocessor 122 will determine the deviation caused by the difference between the optimal reaction temperature and the practical temperature using the temperature compensation module 300 and then show the resultant information on the monitor 123 in forms of graphics or light signals. After the test is completed, the used electrode strip 121 is thrown away.

The present invention has been described above with reference to preferred embodiments. However, those skilled in the art will understand that the scope of the present invention need not be limited to the disclosed preferred embodiments. On the contrary, it is intended to cover various modifications and equivalent arrangements within the scope defined in the following appended claims. The scope of the claims should be accorded the broadest interpretation so as to encompass all such modifications and equivalent arrangements.

What is claimed is:

1. A method for determining an amount of an enzyme inhibitor within a sample, comprising:
(a) providing a first enzyme having a dosage for one time use;
(b) providing a reference sample including said enzyme inhibitor of a given amount, said enzyme inhibitor capable of inhibiting a catalytic reaction of said first enzyme;
(c) providing an electrode strip coated with reactants having a dosage for one time use, said reactants including a first compound;
(d) mixing said first enzyme and said reference sample to form a solution, and then applying said solution to said electrode strip to contact said first enzyme with said first compound;
(e) applying a voltage to said electrode strip, and measuring a reference electric current;
(f) providing said sample, and using said sample instead of said reference sample to repeat steps (a) to (e) and obtain an electric current; and
(g) determining the amount of said enzyme inhibitor within said sample according to said reference electric current and said electric current.

2. The method of claim 1, the step of contacting said first enzyme with said first compound produces a second compound.

3. The method of claim 2, wherein said reactants includes a third compound reacting with said second compound to produce a fourth compound, and said voltage causes said fourth compound to generate said reference electric current and said electric current.

4. The method of claim 3, further comprising providing a second enzyme having a dosage for one time use to catalyze a reaction of said third compound and said second compound.

5. The method of claim 3, further comprising coating said third compound on said electrode strip.

6. The method of claim 4, further comprising coating said second enzyme on said electrode strip.

7. The method of claim 1, wherein said enzyme inhibitor is selected from the group consisting of organophosphorus, thio-organophosphorus treated by a bromide water, and carbamate.

8. The method of claim 1, wherein said first compound is acetylcholine (ACh) or acetylthiocholine (ATC), and said first enzyme is acetylcholine esterase (AChE).

9. The method of claim 1, wherein said step (a) further includes providing a buffer salt.

10. The method of claim 3, wherein said third compound is oxygen from the atmosphere.

11. The method of claim 3, wherein said third compound is ferric cyanide.

12. An apparatus for determining an amount of an enzyme inhibitor within a sample, comprising:
an electrode strip, including an insulation substrate and reactants having a dosage for one time use on said insulation substrate, said reactants including a first compound; and
a microprocessor coupling with said electrode strip;
wherein when said sample and a first enzyme contact with said first compound, an electric signal is generated, and said microprocessor processes said electric signal to determine said amount of said enzyme inhibitor inhibiting said first enzyme.

13. The apparatus of claim 12, wherein when said first enzyme contacts with said first compound, a second compound is produced.

14. The apparatus of claim 13, wherein said reactants includes a third compound reacting with said second compound to produce a fourth compound, and said electric signal is produced by said fourth compound.

15. The apparatus of claim 14, further comprising a second enzyme having a dosage for one time use on said electrode strip, wherein said second enzyme catalyzes a reaction of said third compound and said second compound.

16. The apparatus of claim 12, wherein said first compound is acetylcholine (ACh) or acetylthiocholine (ATC), said first enzyme is acetylcholine esterase (AChE).

17. The apparatus of claim 14, wherein said third compound is ferric cyanide.

18. The apparatus of claim 12, further comprising a temperature control component.

19. The apparatus of claim 12, further comprising a temperature compensation module.
